(19) United States
(12) Patent Application Publication
(10) Pub. No.: US 2012/0329057 A1
(43) Pub. Date: Dec. 27, 2012

(54) BEEF-SPECIFIC AGE DETERMINATION MARKER CONTAINING THE P21 PROTEIN

(75) Inventors: Ik Soon Jang, Seoul (KR); Jong Soon Choi, Daejeon (KR); Joseph Kwon, Jeonju-si (KR); Dong-Gi Lee, Daejeon (KR); Kyeong Eun Yang, Gwangju (KR)

(73) Assignee: KOREA BASIC SCIENCE INSTITUTE, Daejeon (KR)

(21) Appl. No.: 13/519,769

(22) PCT Filed: Dec. 24, 2010
(86) PCT No.: PCT/KR10/09307

§ 371 Date: Jun. 28, 2012

(30) Foreign Application Priority Data
Dec. 30, 2009 (KR) 10-2009-0134212

(51) Int. Cl. G01N 33/566; G01N 21/78; C07K 16/18; G01N 21/64; C07K 14/47; C12Q 1/68; G01N 27/62

(52) U.S. Cl. 435/6.12; 435/7.1; 435/7.92; 435/29; 435/7.94; 530/350; 530/389.8

(57) ABSTRACT

The present invention relates to a beef-specific age determination marker containing the p21 protein, to a beef-specific age determination kit containing an antibody which is specifically bound to the p21 protein, and to a method which involves detecting the p21 protein through an antigen-antibody binding reaction using an antibody which is specifically bound to the p21 protein serving as a beef-specific age determination marker in the muscle tissue of beef, so as to determine the age of the beef. According to the present invention, the p21 protein is significantly greatly expressed in the muscle tissue of beef, the age of which is lower than 30 months, and is hardly expressed in the muscle tissue of beef, the age of which is greater than 30 months, and thus can be valuably used as a beef-specific age determination marker.

### TECHNICAL FIELD

The present invention relates to a marker for determining the age of beef, comprising protein p21 specific for beef, a beef-specific kit for determining the age of beef, comprising an antibody specifically binding to protein p21, and a method for determining beef age, comprising determining the age of beef, using an antigen-antibody binding reaction in which the muscle level of protein p21 is quantitatively analyzed with an antibody specific to p21.

### BACKGROUND ART

With the opening up of the domestic market to beef, South Korea imports a great deal of beef from Canada, the U.S. and other foreign countries. Of the beef imported from North America, such as Canada and the U.S., however, much is from cattle aged over 30 months, which is at great risk for bovine spongiform encephalopathy (BSE), evoking a national concern in South Korea. In fact, 95% of cattle with BSE are known to be over 30 months of age. It is thus required to import beef of less than 30 months of age. However, it is not easy to accurately determine the age of beef.

Currently, the age of beef is determined 1) by a birth certificate or estimated by 2) evaluating the ossification of the bones along the split vertebral column of the carcass or 3) counting the number of permanent incisor teeth in cattle at slaughter. Of these, the most reliable method is the determination made using a birth certificate. On American farms, however, the cattle are generally put out to pasture, so that their birthdays are not accurate. In fact, determining the age using the birth certificate is possible only in as few as 20% of the cattle imported from the U.S. Estimating the age by examining the ossification of the bones, that is, physiological skeletal maturity, is regarded as the most reliable among age determination methods with the naked eye, thus far, but the error rate is as large as about 15%. In addition, the number of teeth in cattle at slaughter is not scientifically accepted as an index for age estimation because the number of teeth greatly varies depending on breeding conditions. Particularly, the teeth-counting method is less accurate for cattle that are put out to pasture because they are not under regulated feeding conditions.

As stated above, currently used methods of determining the age of beef are not accurate, except for using the birth certificate.

Thanks to much study that has been done into determining the origin, grade and maturity of beef, Korean native and imported cattle carcasses can be classified in detail according to the origin, grade and maturity, but the exact age thereof cannot be determined. In addition, examining imported beef using currently used methods requires a great deal of time and expense because of the tremendous amount of such beef.

Therefore, there is a need for a scientific method for accurately determining the age of beef at low cost in a reduced period of time.

### DISCLOSURE

### Technical Problem

Cumulating in the present invention, intensive and thorough research into accurately determining the age of beef, conducted by the present inventors, led to the finding that protein p21 is expressed at a high level in the muscle of cattle less than 30 months old, but almost not expressed in the muscles of cattle over 30 months of age.

### Technical Solution

It is an object of the present invention to provide a beef-specific marker for determining the age of beef, comprising protein p21.

It is another object of the present invention to provide a beef-specific kit for determining the age of beef, comprising an antibody specifically binding to protein p21.

It is a further object of the present invention to provide a method for determining the age of beef, using an antigen-antibody binding reaction in which the muscle level of protein p21 is quantitatively analyzed with an antibody specific to p21.

### DESCRIPTION OF DRAWINGS

FIG. 1 shows expression levels of p53 and p21 in the muscle tissue of beef, as measured by Western blot.

FIG. 2 shows expression levels of p53 and p21 in the dermal tissue of mice, as measured by Western blot.

### BEST MODE

In accordance with an aspect thereof, the present invention addresses a beef-specific marker for determining the age of beef, comprising protein p21.

In accordance with another aspect thereof, the present invention addresses a beef-specific kit for determining the age of beef, comprising an antibody specifically binding to protein p21.

In accordance with a further aspect thereof, the present invention addresses a method for determining the age of beef, comprising detecting the muscle level of protein p21 by conducting an antigen-antibody binding reaction in which the protein p21, useful as a beef specific marker, is reacted with an antibody specific to p21.

Below, a detailed description will be given of the present invention.

Protein p21 is detected at a very high level in muscle tissues of cattle below 30 months of age, but almost no levels are found in muscle tissue of cattle over 30 months of age. In detail, beef is determined to be younger than 30 months when the expression level ratio of p21 to GAPDH is over 0.5 in the muscle tissue of beef while being older than 30 months of age in the muscle tissue of beef when the expression level ratio of p21 to GAPDH is below 0.5.

The expression of the p21 gene is tightly controlled by p53 because p21 is a transcriptional target of the tumor suppressor gene p53. p21, also known as CDK (cyclin-dependent kinase) inhibitor, functions as a regulator of cell cycle. The protein is encoded by the CDKN1A gene located on chromosome 6 (6p21.2) in humans. In the muscle tissue of cattle aged below 30 months, no p53 proteins are expressed. In contrast, a high expression level of p53 is detected in the muscle tissue of cattle aged over 30 months. In mice, further, expression levels of both p53 and p21 are found to increase with age. Therefore, protein p21 can be used as a beef-specific marker for the determination of age.

The beef-specific marker based on protein p21 in accordance with the present invention makes it easy to determine the age of beef, which is difficult to determine using conventional methods. Thus, it can be used as an index for cattle age. Further, the recruitment of the beef-specific marker in accordance with the present invention for the determination of age can significantly reduce the time and expense of performing a quarantine inspection on imported beef, and guarantee more reliable inspection, compared to conventional methods.

The beef-specific kit for the determination of age of beef comprising an antibody specifically binding to protein p21 in accordance with the present invention can be readily prepared on the basis of the marker using a typical method known in the art.

In one embodiment of the present invention, the beef-specific kit may comprise an antibody specifically binding to protein p21, a secondary antibody conjugate with a label that can react with a substrate to cause a chromatic change; a substrate solution which develops a color upon reaction with the label; a washing buffer and a reaction stop buffer.

The label conjugated to the secondary antibody is preferably a coloring agent which can bring about a color change as it reacts with its substrate. Representative among them are HRP (horseradish peroxidase), alkaline phosphatase, colloid gold, fluorescein such as FITC (poly L-lysine-fluorescein isothiocyanate) and RITC (rhodamine-B-isothiocyanate), and dye.

As for the substrate solution, it is dependent on the label. Examples include TMB (3,3′,5,5′-tetramethyl bezidine), ABTS [2,2′-azino-bis(3-ethylbenzothiazoline-6-sulfonic acid)], and OPD (o-phenylenediamine). The coloring substrate is preferably provided in the form of a solution in buffer (0.1M NaOAc, pH 5.5).

The washing solution preferably contains phosphate buffer, NaCl and Tween 20. After the antibody is allowed to react with the antigen, the antigen-antibody complex is treated with the secondary antibody conjugate, followed by immobilization and then washing 3˜6 times with the washing solution. A sulfuric acid solution may be used to stop the enzymatic reaction.

In one embodiment of the present invention, the age of beef can be determined using an antigen-antibody binding reaction in which the muscle level of protein p21, useful as a beef-specific marker, is quantitatively analyzed with an antibody specific to p21. In greater detail, protein p21 is separated by SDS-PAGE, transferred and fixed onto an immobilizer which is then treated with an antibody against protein p21 to form an antigen-antibody complex which is useful for determining the expression level of protein p21. That is, the age of beef can be judged to be below 30 months when the relative expression level of protein p21 to GAPDH in the muscle tissue is over 0.5 and to be over 30 months when the relative expression level is below 0.5.

As the immobilizer useful in the antigen-antibody binding reaction, a nitrocellulose membrane, a PVDF (polyvinylidene difluoride) membrane, a 96-well plate formed of polyvinyl resin or polystyrene resin, or a slide glass may be used.

The antigen-antibody binding reaction may be assayed using a typical method such as enzyme-linked immunosorbent assay (ELISA), radioimmunoassay (RIA), sandwich assay, reverse-transcription polymerase chain reaction (RT-PCR), realtime PCR, Western blot, immunoprecipitation, immunohistochemical staining, tandem mass spectrometry (LC-MS/MS), immunofluorescence assay, enzyme-substrate coloring assay, antigen-antibody aggregation.

### Mode for Invention

A better understanding of the present invention may be obtained through the following examples which are set forth to illustrate, but are not to be construed as limiting the present invention.

### Example 1

### Determination of Beef Age—Western Blot

Three cattle per group of naturally aged 20, 28, 31, 35 and 38 month old cattle were used. They were slaughtered and their beef muscle tissues were analyzed for protein level with respect to age. In more detail, the beef muscle tissues were homogenized in buffer [20 mM Tris-HCl, PIC (protease inhibitor cocktail, Roche)], left for 30 min on ice, and centrifuged for min at 12,000 rpm at 4° C. The protein level of the supernatant thus obtained was determined by the BCL method. The same protein amounts were mixed with 5×SDS sample buffer [60 mM Tris-Cl (pH 6.8), 25% glycerol, 2% SDS, 14.4 mM β-mercaptoethanol, 0.1% bromophenol blue] and boiled for 5 min. The proteins thus denatured (30˜40 μg) were run on 12% polyacrylamide gel by electrophoresis and then transferred onto a nitrocellulose membrane. In this regard, electrophoresis was performed on a 0.2 μm nitrocellulose membrane in transfer buffer [25 mM Tris-base (pH 8.3), 192 mM glycine, 20% methanol] for 2 hours under an electric field of 1 A. Thereafter, the nitrocellulose membrane was stained with Ponceau to determine protein positions. The membrane was blocked for one hour with 5% skimmed milk in TTBS (Tris Buffered Saline with Tween 20), followed by incubation with a dilution of primary antibodies (p21, p53) at room temperature for 2 hours or at 4° C. overnight. Then, the membrane was washed three times for 5 min with TBS-0.1% Tween 20 before incubation with a 1:2000 dilution of HRP-conjugated goat anti-mouse IgG or HRP-conjugated rabbit anti-goat IgG in TBS containing 5% skimmed milk at room temperature for one hour. Again, the membrane was rinsed three times for 5 min with TBS-0.1% Tween 20, after which an ECL kit (Pierce) containing a peroxidase substrate was used to develop the proteins on an X-ray film (Kodak). Also, relative expression levels of p21 (to GAPDH) were quantitatively analyzed using a densitometer.

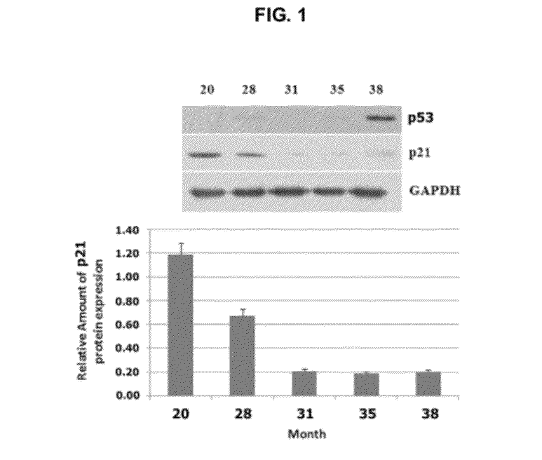

The results are shown in FIG. 1.

As can be seen in FIG. 1, protein p53 was expressed in the muscle tissues of beef only from cattle aged 38 months whereas almost no protein p21 was found in cattle over 30 months of age. In addition, the relative expression level of p21 to GAPDH was found to be greater than 0.5 in the muscle tissues of cattle below 30 months of age, and to be below 0.5 in the muscle tissues of cattle over 30 months of age. Therefore, protein p21 can be used as a beef-specific marker for determining the age of cattle.

### Comparative Example 1

### Determination of Mouse Age Western Blot

C57BL/6 mice were bred in an SPF (specific pathogen free) facility for up to 24 months. A selection was made of mice aged 3, 6, 9, 12, 15, 18, 21, and 24 months. After no clear diseases were found in appearance and anatomically from the mice, organs were excised therefrom. Dermal tissues with a dimension of about 1 cm×1 cm were frozen in liquid nitrogen and milled in a mortar. The milled dermal tissues were dissolved in RIPA buffer [5 mM Tris-Cl (pH 7.4), 1% NP-40, 0.25% sodium deoxycholate, 150 mM NaCl, 1 mM EDTA] by ultrasonication. Two rounds of centrifugation at 14,000×g for 10 min separated proteins in a supernatant. SDS-PAGE was performed with 20 mg of the proteins thus obtained. As a secondary antibody, an anti-mouse antibody (Amersham, Chicago, Ill.) was diluted 1:10,000 before incubation with the proteins. Color development was achieved using ECL (enhanced chemiluminescence system; Amersham).

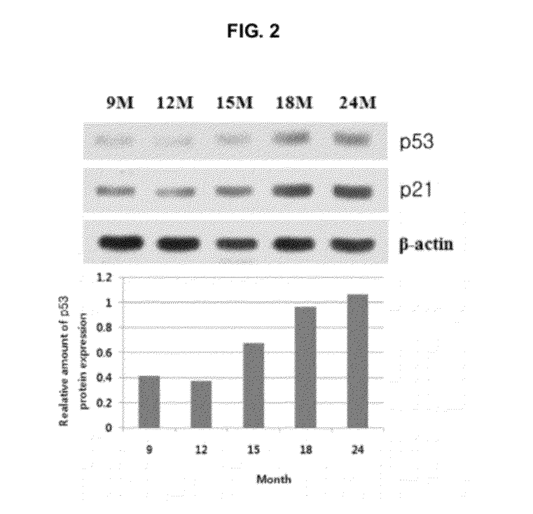

The results are shown in FIG. 2.

As can be seen in FIG. 2, the expression levels of p53 and p21 in the dermal tissue of mice were found to increase with age.

### INDUSTRIAL APPLICABILITY

Because protein p21 is expressed at a high level in the muscle tissue of cattle below 30 months of age, but not at all in the muscle tissue of cattle over 30 months of age, as described hitherto, the protein p21 can be used as a beef-specific marker for the determination of age of beef. The beef-specific marker based on protein p21 in accordance with the present invention makes it easy to determine the age of beef, which is difficult to determine using conventional methods, so that it can be used as an index for cattle age. Further, the recruitment of the beef-specific marker in accordance with the present invention for the determination of age can significantly reduce the time and expense that is taken up by performing a quarantine inspection on imported beef, and guarantee more reliable inspection, compared to conventional methods.

1. A beef-specific age determination marker, comprising protein p21.

2. A beef-specific age determination kit, comprising an antibody that specifically binding to protein p21.

3. A method for determining age of beef, comprising detecting a level of protein p21 in muscle by carrying out an antigen-antibody binding reaction in which the antibody is specifically binding to protein p21.

4. The method according to claim 3, wherein the antigen-antibody binding reaction is performed using a method selected from the group consisting of enzyme-linked immunosorbent assay (ELISA), radioimmunoassay (RIA), sandwich assay, reverse-transcription polymerase chain reaction (RT-PCR), realtime PCR, Western blot, immunoprecipitation, immunohistochemical staining, tandem mass spectrometry (LC-MS/MS), immunofluorescence assay, enzyme-substrate coloring assay, antigen-antibody aggregation, and a combination thereof.

5. The method according to claim 3, wherein the age of beef is determined to be less than 30 months when an expression level ratio of p21 to GAPDH in a muscle tissue of the beef is greater than 0.5, and determined to be over 30 months when an expression level ratio of p21 to GAPDH in a muscle tissue of the beef is below 0.5.
